(19) United States
(12) Patent Application Publication
(10) Pub. No.: US 2010/0228310 A1
(43) Pub. Date: Sep. 9, 2010

(54) SYSTEMS AND METHODS FOR AUTONOMIC NERVE MODULATION

(76) Inventors: Allan C. Shuros, St. Paul, MN (US); Weiying Zhao, Cupertino, CA (US); Jason J. Hamann, Blaine, MN (US); Stephen Ruble, Lino Lakes, MN (US)

Correspondence Address: SCHWEGMAN, LUNDBERG & WOESSNER/BSC-CRM, PO BOX 2938, MINNEAPOLIS, MN, 55402, US

(21) Appl. No.: 12/715,862

(22) Filed: Mar. 2, 2010

Related U.S. Application Data
(60) Provisional application No. 61/158,623, filed on Mar. 9, 2009.

(51) Int. Cl. A61N 1/36

(52) U.S. Cl. 607/17; 607/72; 607/62

(57) ABSTRACT

According to various embodiments of a method for modulating autonomic neural activity in a body having a spinal cord, a subclavian vein and thoracic lymphatic vessels that include a thoracic duct and a right lymphatic duct, at least one programmed therapy is implemented using an implanted medical device to modulate autonomic neural activity. Implementing the therapy includes increasing or decreasing sympathetic activity in sympathetic nerves branching from a first region of the spinal cord using a first electrode in the thoracic duct, and further includes increasing or decreasing parasympathetic activity in parasympathetic nerves adjacent to the desired thoracic lymphatic vessel or sympathetic activity in sympathetic nerves branching from a second region of the spinal cord using a second electrode in the desired thoracic lymphatic vessel.

### CROSS-REFERENCE TO RELATED APPLICATION

This application claims the benefit of U.S. Provisional Application No. 61/158,623, filed on Mar. 9, 2009, under 35 U.S.C. §119(e), which is hereby incorporated by reference in its entirety.

### TECHNICAL FIELD

This application relates generally to medical devices and, more particularly, to systems, devices and methods for modulating the autonomic nervous system.

### BACKGROUND

Neural stimulation has been applied to treat various pathological conditions. Controlled delivery of electrical stimulation pulses to a nerve generates, modulates, or inhibits activities of that nerve, thereby restoring the functions of that nerve and/or regulating the functions of the tissue or organ innervated by that nerve. One specific example of neural stimulation is to regulate cardiac functions and hemodynamic performance by delivering electrical stimulation pulses to portions of the autonomic nervous system. The heart is innervated with sympathetic and parasympathetic nerves.

Therapies that are based on autonomic modulation have shown efficacy in a variety of cardiovascular diseases in both preclinical and clinical studies. The autonomic balance can be modulated to have more parasympathetic tone by stimulating parasympathetic targets or inhibiting sympathetic targets, and can be modulated to have more sympathetic tone by stimulating sympathetic targets or inhibiting parasympathetic targets.

Autonomic imbalance is associated with a number of cardiac and other diseases (heart failure (HF), coronary artery disease (CAD), inflammation, diabetes, obesity, epilepsy, depression, etc.). Some neural stimulation systems place electrodes on specific nerves. Spinal cord stimulation has been proposed, but is not able to target the vagus nerve directly.

### SUMMARY

Various system embodiments modulate autonomic neural activity in a body having a spinal cord, a subclavian vein and thoracic lymphatic vessels that include a thoracic duct and a right lymphatic duct. According to various embodiments, the system includes a programmable neural stimulator and at least one stimulation lead. The lead(s) includes a first electrode region and a second electrode region, and is adapted to be fed through the subclavian vein into a desired thoracic lymphatic vessel to operationally position the first electrode region in the thoracic lymphatic vessel to stimulate sympathetic nerves branching from a first region of the spinal cord and to operationally position the second electrode region in the desired thoracic lymphatic vessel to stimulate sympathetic nerves branching from a second region of the spinal cord or stimulate parasympathetic nerves anatomically adjacent to the desired thoracic lymphatic vessel. The neural stimulator is programmed to deliver neural stimulation pulses to the first electrode region to modulate sympathetic activity in the sympathetic nerves branching from the first region of the spinal cord and to deliver neural stimulation pulses to the second electrode region to modulate sympathetic activity in the sympathetic nerves branching from the second region of the spinal cord or to modulate parasympathetic activity in the parasympathetic nerves anatomically adjacent to the desired thoracic lymphatic vessel.

According to various embodiments of a method for modulating autonomic neural activity in a body having a spinal cord, a subclavian vein and thoracic lymphatic vessels that include a thoracic duct and a right lymphatic duct, at least one programmed therapy is implemented using an implanted medical device to modulate autonomic neural activity. Implementing the therapy includes increasing or decreasing sympathetic activity in sympathetic nerves branching from a first region of the spinal cord using a first electrode in the thoracic duct, and further includes increasing or decreasing parasympathetic activity in parasympathetic nerves adjacent to the desired thoracic lymphatic vessel or sympathetic activity in sympathetic nerves branching from a second region of the spinal cord using a second electrode in the desired thoracic lymphatic vessel.

According to various embodiments of a method for implanting a system for modulating both parasympathetic and sympathetic activity in a body having a spinal cord, a subclavian vein and thoracic lymphatic vessels that include a thoracic duct and a right lymphatic duct, at least one stimulation lead is fed through the subclavian vein into the thoracic lymphatic vessels to operationally position a first electrode region in the thoracic duct to stimulate sympathetic nerves branching from a first region of the spinal cord and to operationally position a second electrode region in the thoracic lymphatic vessels to stimulate parasympathetic nerves adjacent to the desired thoracic lymphatic vessel. A programmable neural stimulator is implanted and is operationally attached to the at least one stimulation lead to stimulate the sympathetic nerves and parasympathetic nerves. A test routine is implemented to verify capture of the sympathetic nerves and parasympathetic nerves.

This Summary is an overview of some of the teachings of the present application and not intended to be an exclusive or exhaustive treatment of the present subject matter. Further details about the present subject matter are found in the detailed description and appended claims. Other aspects will be apparent to persons skilled in the art upon reading and understanding the following detailed description and viewing the drawings that form a part thereof, each of which are not to be taken in a limiting sense. The scope of the present invention is defined by the appended claims and their equivalents.

### BRIEF DESCRIPTION OF THE DRAWINGS

FIG. 1 illustrates an embodiment of a neural stimulation system and portions of an environment in which the system is used.

FIG. 2 illustrates an embodiment of the neural stimulation system.

FIGS. 3A-3C illustrate anatomy proximate to the thoracic duct.

FIGS. 4-7B illustrate various lead embodiments.

FIG. 8 illustrates a method embodiment to implant the spinal cord stimulation lead to establish and maintain efficacious stimulation therapy.

FIGS. 9-18 illustrate various algorithms that can be programmed into the implantable device to control the translymphatic stimulation of the sympathetic and parasympathetic nerves.

FIG. 19 shows a system diagram of an embodiment of a microprocessor-based implantable device, according to various embodiments.

FIG. 20 illustrates a system including an external device, an implantable neural stimulator (NS) device and an implantable cardiac rhythm management (CRM) device, according to various embodiments of the present subject matter.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

The present subject provides a minimally invasive method and apparatus for strategic ANS modulation via the thoracic duct of the lymphatic system. The thoracic duct of the lymphatic system lies in proximity to the vagus nerve and sympathetic nerves branching from the spinal column and offers a minimally invasive approach to stimulating these structures. Embodiments of the present subject matter provides a means to stimulate (increase or decrease activity) both sympathetic and parasympathetic nerves, without the need for two separate surgery sites and separate leads.

According to various embodiments for modulating the sympathetic nervous system, a lead has a first set of electrodes and a second set of electrodes contained on the same lead body (or a telescoping outer body member) to target sympathetic nerves branching from spinal cord in a first region using the first set of electrodes and to target sympathetic nerves branching from the spinal cord in a second region using the second set of electrodes. According to various embodiments, the first and second regions of the spinal cord are in the thoracic and/or cervical regions of the spinal cord. In humans, nerves branching generally from the spinal cord in the C5-C7 region and nerves branching generally from the spinal cord in the T1-T6 region innervate the heart and can affect cardiovascular performance. By way of example and not limitation, the first and second electrodes can be positioned to stimulate nerves branching from the spinal cord in the C7/T1 region and nerves branching from the spinal cord in the T4/T5 region. The nerves branching from these different regions of the spinal cord innervate different areas or innervate areas to a greater or lesser extent. Thus, stimulation of nerves in these different regions may modulate sympathetic tone in different areas, or may modulate sympathetic tone in the same area to different extents.

According to various embodiments for modulating both the parasympathetic and the sympathetic nervous system, a lead has a first set of electrodes and a second set of electrodes contained on the same lead body (or a telescoping outer body member) to target sympathetic nerves branching from spinal cord in a first region using the first set of electrodes and to target parasympathetic nerves (e.g. the vagus nerve originating from the medulla oblongata) lying anatomically adjacent to the thoracic duct in the cervical and intrathoracic inlet region.

As will be understood by those of ordinary skill in the art, a neural target can be stimulated with a set of parameters to increase or elicit neural activity in the nerve, and can be stimulated with another set of parameters to decrease, inhibit or block neural activity in the nerve. Thus, various embodiments provide a programmable neural stimulator that is programmed to deliver neural stimulation pulses to decrease sympathetic activity in the sympathetic nerves branching from the spinal cord and to deliver neural stimulation pulses to increase sympathetic activity in the sympathetic nerves branching from the spinal cord; and various embodiments provide a programmable neural stimulator that is programmed to deliver neural stimulation pulses to decrease parasympathetic activity in the parasympathetic nerves adjacent to the thoracic duct and to deliver neural stimulation pulses to increase parasympathetic activity in the parasympathetic nerves adjacent to the thoracic duct.

In an embodiment, the programmable neural stimulator is programmed to chronically deliver neural stimulation pulses to chronically inhibit sympathetic activity in the sympathetic nerves branching from the spinal cord, and to intermittently deliver neural stimulation pulses to intermittently increase parasympathetic activity in the parasympathetic nerves (e.g. vagus nerve) adjacent to the thoracic duct.

In an embodiment, the programmable neural stimulator is programmed to chronically deliver neural stimulation pulses to increase sympathetic activity in the sympathetic nerves branching from the spinal cord, and intermittently or chronically deliver neural stimulation pulses to increase parasympathetic activity in the parasympathetic nerves (e.g. vagus nerve) adjacent to the thoracic duct. An embodiment chronically-delivers low-level sympathetic activation to enhance the impact of vagal stimulation through a vagal-sympathetic accentuated antagonism effect.

In an embodiment, the system includes a respiratory sensor, and the programmable neural stimulator is programmed to time delivery of neural stimulation pulses to decrease sympathetic activity during the inspiratory phase, and time delivery of neural stimulation pulses to increase parasympathetic activity during the expiratory phase. The respiration sensor can be used to guide the neural stimulation to block sympathetic activity during the inspiratory phase when sympathetic activity is intrinsically high, and to stimulate the vagus nerve during an expiratory phase to enhance the parasympathetic activity.

In an embodiment, the system includes an arrhythmia detector used to detect a cardiac arrhythmia, and the programmable neural stimulator is programmed to implement an anti-arrhythmia therapy by delivering neural stimulation pulses to decrease sympathetic activity in the sympathetic nerves branching from the spinal cord if the arrhythmia detector detects the cardiac arrhythmia, and implement a chronic heart failure therapy by delivering neural stimulation pulses to chronically increase parasympathetic activity in the parasympathetic nerves (e.g. vagus nerve) adjacent to the thoracic duct. This embodiment can be combined with various cardiac rhythm management devices (e.g. implantable cardioverter defibrillator) that detect and treat arrhythmias.

In an embodiment, the programmable neural stimulator is programmed to deliver neural stimulation pulses to increase sympathetic activity in the sympathetic nerves branching from the spinal cord, deliver neural stimulation pulses to increase parasympathetic activity in the parasympathetic nerves (e.g. vagus nerve) adjacent to the thoracic duct, and control timing of the neural stimulation pulses to intermittently increase both sympathetic and parasympathetic activity, and to follow increased sympathetic activity with increased parasympathetic activity. For example, sympathetic stimulation is delivered for a period of time. After the sympathetic stimulation ends, there is an intrinsic parasympathetic reflex response, which is augmented using vagal stimulation.

According to various embodiments, the translymphatic stimulation can be delivered using a single lead or using multiple leads. Some embodiments of the present subject matter provide a lead with two electrode sets to target two different neural targets for translymphatic stimulation. Some embodiments provide the lead with a telescoping capability to allow the distance between the electrode or electrode sets to be varied during implantation. Appropriate neural capture is ensured during the implantation process by monitoring heart rate or contractility or respiration or blood pressure to ensure capture. Multiple electrodes (e.g. tripolar or quadripolar designs) can be used to individually steer.

### Physiology

Provided below is a brief discussion of some diseases capable of being treated using the present subject matter and of the nervous system. This discussion is believed to assist a reader in understanding the disclosed subject matter.

### Diseases

The present subject matter can be used to prophylactically or therapeutically treat various diseases by modulating autonomic tone. Examples of such diseases or conditions include hypertension, cardiac remodeling, and heart failure.

Hypertension is a cause of heart disease and other related cardiac co-morbidities. Hypertension occurs when blood vessels constrict. As a result, the heart works harder to maintain flow at a higher blood pressure, which can contribute to heart failure. Hypertension generally relates to high blood pressure, such as a transitory or sustained elevation of systemic arterial blood pressure to a level that is likely to induce cardiovascular damage or other adverse consequences. Hypertension has been defined as a systolic blood pressure above 140 mm Hg or a diastolic blood pressure above 90 mm Hg. Consequences of uncontrolled hypertension include, but are not limited to, retinal vascular disease and stroke, left ventricular hypertrophy and failure, myocardial infarction, dissecting aneurysm, and renovascular disease. A large segment of the general population, as well as a large segment of patients implanted with pacemakers or defibrillators, suffer from hypertension. The long term mortality as well as the quality of life can be improved for this population if blood pressure and hypertension can be reduced. Many patients who suffer from hypertension do not respond to treatment, such as treatments related to lifestyle changes and hypertension drugs.

Following myocardial infarction (MI) or other cause of decreased cardiac output, a complex remodeling process of the ventricles occurs that involves structural, biochemical, neurohormonal, and electrophysiologic factors. Ventricular remodeling is triggered by a physiological compensatory mechanism that acts to increase cardiac output due to so-called backward failure which increases the diastolic filling pressure of the ventricles and thereby increases the so-called preload (i.e., the degree to which the ventricles are stretched by the volume of blood in the ventricles at the end of diastole). An increase in preload causes an increase in stroke volume during systole, a phenomena known as the Frank-Starling principle. When the ventricles are stretched due to the increased preload over a period of time, however, the ventricles become dilated. The enlargement of the ventricular volume causes increased ventricular wall stress at a given systolic pressure. Along with the increased pressure-volume work done by the ventricle, this acts as a stimulus for hypertrophy of the ventricular myocardium. The disadvantage of dilatation is the extra workload imposed on normal, residual myocardium and the increase in wall tension (Laplace's Law) which represent the stimulus for hypertrophy. If hypertrophy is not adequate to match increased tension, a vicious cycle ensues which causes further and progressive dilatation. As the heart begins to dilate, afferent baroreceptor and cardiopulmonary receptor signals are sent to the vasomotor central nervous system control center, which responds with hormonal secretion and sympathetic discharge. It is the combination of hemodynamic, sympathetic nervous system and hormonal alterations (such as presence or absence of angiotensin converting enzyme (ACE) activity) that ultimately account for the deleterious alterations in cell structure involved in ventricular remodeling. The sustained stresses causing hypertrophy induce apoptosis (i.e., programmed cell death) of cardiac muscle cells and eventual wall thinning which causes further deterioration in cardiac function. Thus, although ventricular dilation and hypertrophy may at first be compensatory and increase cardiac output, the processes ultimately result in both systolic and diastolic dysfunction (decompensation). It has been shown that the extent of ventricular remodeling is positively correlated with increased mortality in post-MI and heart failure patients.

Heart failure (HF) refers to a clinical syndrome in which cardiac function causes a below normal cardiac output that can fall below a level adequate to meet the metabolic demand of peripheral tissues. Heart failure may present itself as congestive heart failure (CHF) due to the accompanying venous and pulmonary congestion. Heart failure can be due to a variety of etiologies such as ischemic heart disease. Heart failure patients have reduced autonomic balance, which is associated with LV dysfunction and increased mortality.

### Nervous System

The autonomic nervous system (ANS) regulates “involuntary” organs, while the contraction of voluntary (skeletal) muscles is controlled by somatic motor nerves. Examples of involuntary organs include respiratory and digestive organs, and also include blood vessels and the heart. Often, the ANS functions in an involuntary, reflexive manner to regulate glands, to regulate muscles in the skin, eye, stomach, intestines and bladder, and to regulate cardiac muscle and the muscle around blood vessels, for example.

The ANS includes the sympathetic nervous system and the parasympathetic nervous system. The sympathetic nervous system is affiliated with stress and the “fight or flight response” to emergencies. Among other effects, the “fight or flight response” increases blood pressure and heart rate to increase skeletal muscle blood flow, and decreases digestion to provide the energy for “fighting or fleeing.” The parasympathetic nervous system is affiliated with relaxation and the “rest and digest response” which, among other effects, decreases blood pressure and heart rate, and increases digestion to conserve energy. The ANS maintains normal internal function and works with the somatic nervous system. Afferent nerves convey impulses toward a nerve center, and efferent nerves convey impulses away from a nerve center.

The heart rate and contractile force is increased when the sympathetic nervous system is stimulated, and is decreased when the sympathetic nervous system is inhibited. The heart rate and force is decreased when the parasympathetic nervous system is stimulated, and is increased when the parasympathetic nervous system is inhibited. Cardiac rate, contractility, and excitability are known to be modulated by centrally mediated reflex pathways. Baroreceptors and chemoreceptors in the heart, great vessels, and lungs, transmit cardiac activity through vagal and sympathetic afferent fibers to the central nervous system. Activation of sympathetic afferents triggers reflex sympathetic activation, parasympathetic inhibition, vasoconstriction, and tachycardia. In contrast, parasympathetic activation results in bradycardia, vasodilation, and inhibition of vasopressin release. Among many other factors, decreased parasympathetic or vagal tone or increased sympathetic tone is associated with various arrhythmias genesis, including ventricular tachycardia and atrial fibrillation. The functions associated with the sympathetic and parasympathetic nervous systems are many and can be complexly integrated with each other.

Neural stimulation can be used to stimulate/increase nerve traffic or inhibit/decrease nerve traffic. An example of neural stimulation to stimulate nerve traffic is a lower frequency signal (e.g. within a range on the order of 20 Hz to 50 Hz). An example of neural stimulation to inhibit nerve traffic is a higher frequency signal (e.g. within a range on the order of 120 Hz to 150 Hz). Other methods for stimulating and inhibiting nerve traffic have been proposed.

Modulation of the autonomic nervous system has potential clinical benefit in preventing remodeling and death in heart failure and post-MI patients. Electrical stimulation can be used to inhibit sympathetic nerve activity and reduce blood pressure by decreasing vascular resistance. Sympathetic inhibition, which increases parasympathetic tone, has been associated with reduced arrhythmia vulnerability following a myocardial infarction, presumably by increasing collateral perfusion of the acutely ischemic myocardium and decreasing myocardial damage.

### System

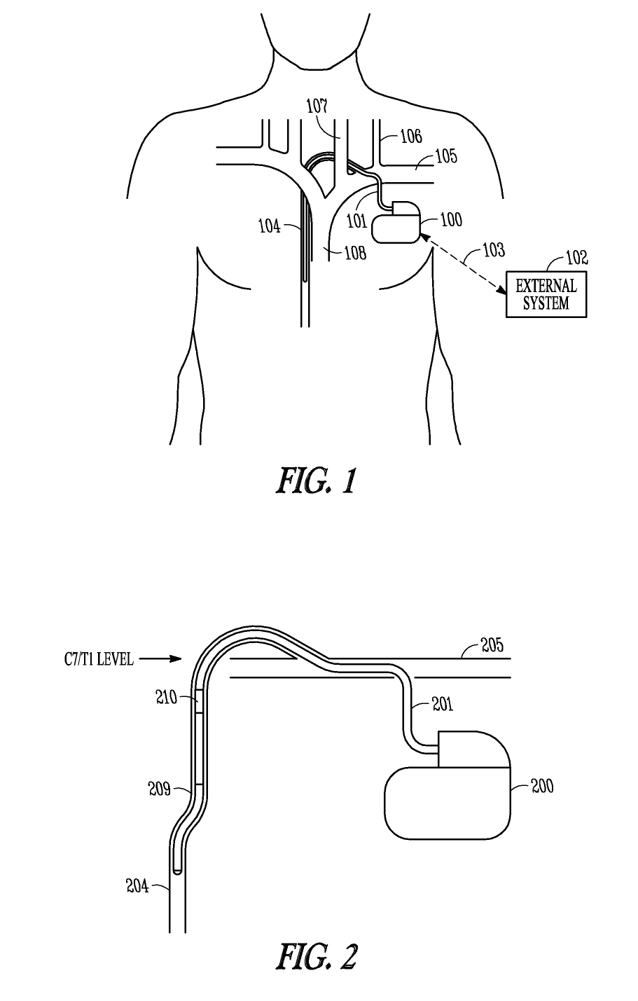

FIG. 1 illustrates an embodiment of a neural stimulation system and portions of an environment in which the system is used. The system includes an implantable medical device 100, a lead 101, an external system 102, and a telemetry link 103 used to communicate between the implantable medical device 100 and the external system 102. Neural stimulation pulses are delivered using at least one electrode placed in a thoracic duct 104, which is part of the lymphatic system of a patient's body. The lymphatic system includes lymph tissue, nodes, and vessels. Interstitial fluid is absorbed from tissue, filtered through lymph nodes, and empties into lymphatic vessels. Most of these vessels from the lower body and left side of the body drain into the thoracic duct which itself typically drains into the left subclavian vein. The right upper quadrant of the body typically drains into the right lymphatic duct which drains into the right subclavian vein. FIG. 1 illustrates portions of the thoracic duct 104, a subclavian vein 105, a left external jugular vein 106, a left internal jugular vein 107, and a superior vena cava 108. The thoracic duct 104 connects to the venous system at the juncture of the subclavian vein 105 and the left internal jugular vein 107. The fluid (lymph) from the lower body flows up to the thoracic duct and empties into the subclavian vein from the thoracic duct. The thoracic duct is located in the posterior mediastinal area of body, adjacent to the heart and various portions of the nervous system including portions of the vagus, sympathetic, and phrenic nerves. Electrical stimulation of such nerves is delivered by using one or more stimulation electrodes placed within the thoracic duct. The thoracic duct is used as a conduit for advancing the stimulation electrode(s) to a location from which electrical stimulation can be delivered to a target of the nervous system. This approach to the process of electrode placement for neural stimulation has the potential of reducing the invasiveness of implantation procedure under many circumstances.

The implantable medical device 100 generates neural stimulation pulses that are electrical pulses and delivers the neural stimulation pulses through the lead 101. In various embodiments, the implantable medical device also senses neural activities or other physiological signals and/or also delivers therapies in addition to the neural stimulation. Examples of such additional therapies include cardiac pacing therapy, cardioversion/defibrillation therapy, cardiac resynchronization therapy (CRT), cardiac remodeling control therapy (RCT), drug therapy, cell therapy, and gene therapy. Some system embodiments provide these functions in a single implantable medical device, and some system embodiments provide these functions using two or more implantable medical devices. In one embodiment, for example, the system includes one or more endocardial and/or epicardial leads for delivering pacing and/or defibrillation pulses to the heart.

The distal portion of the lead is configured for placement in the subclavian vein and the thoracic duct. During the implantation of the lead, the distal end is inserted into the subclavian vein through an incision, advanced in the subclavian vein toward the thoracic duct, inserted into the thoracic duct from the subclavian vein, and advanced in the thoracic duct until a predetermined location in the thoracic duct is reached. In one embodiment, the position of stimulation electrodes is adjusted during implantation by delivering test neural stimulation pulses and detecting evoked neural signals and/or other physiological responses. In one embodiment, the lead includes a fixation mechanism configured to stabilize the distal end in the determined position in the thoracic duct. The implantable medical device is connected to the proximal end and is subcutaneously implanted.

The external system 102 communicates with the implantable medical device 100 and allows a physician or other caregiver to access the implantable medical device. In one embodiment, the external system includes a programmer. In another embodiment, the external system is a patient management system including an external device communicating with an implantable medical device via a telemetry link, a remote device in a relatively distant location, and a telecommunication network linking the external device and the remote device. The patient management system allows access to the implantable medical device from a remote location, for purposes such as monitoring patient status and adjusting therapies. In one embodiment, the telemetry link is an inductive telemetry link. In another embodiment, the telemetry link is a far-field radio-frequency (RF) telemetry link.

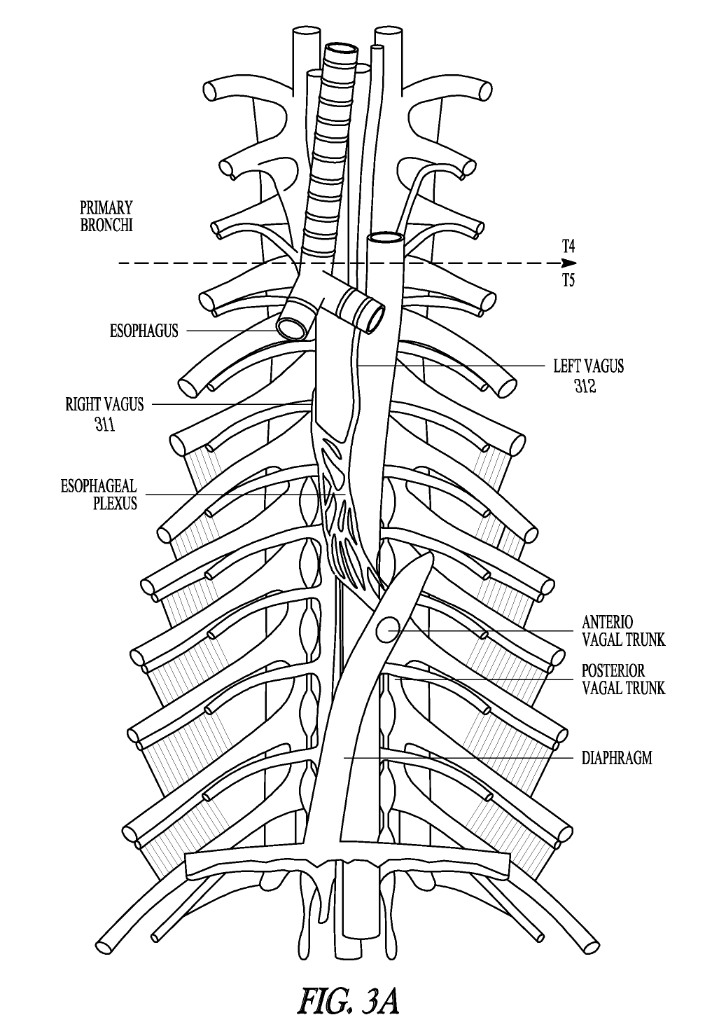

FIG. 2 illustrates an embodiment of the neural stimulation system. The illustrated embodiment includes an implantable medical device 200, and a lead 201 implanted into the thoracic duct 204 via the subclavian vein 205. The illustrated lead includes a first electrode region 209 and a second electrode region 210. The electrode regions include at least one and preferably a plurality of electrodes used to stimulate a neural target. More specifically, in the illustrated embodiment, the first electrode region 209 includes a plurality of electrodes or contacts for use in targeting sympathetic nerves with translymphatic stimulation, and the second electrode region 210 includes a plurality of electrodes or contacts for use in targeting parasympathetic nerves (e.g. vagus nerve(s)) with translymphatic stimulation. Sympathetic nerves can be targeted in the C7/T1 to T5 range, depending on the desired effect. A parasympathetic nerve target is the vagus nerve lying adjacent to the thoracic duct. Some lead embodiments provide a telescoping feature between the first and second electrode regions to allow the distance between these regions to be varied during the implantation procedure.

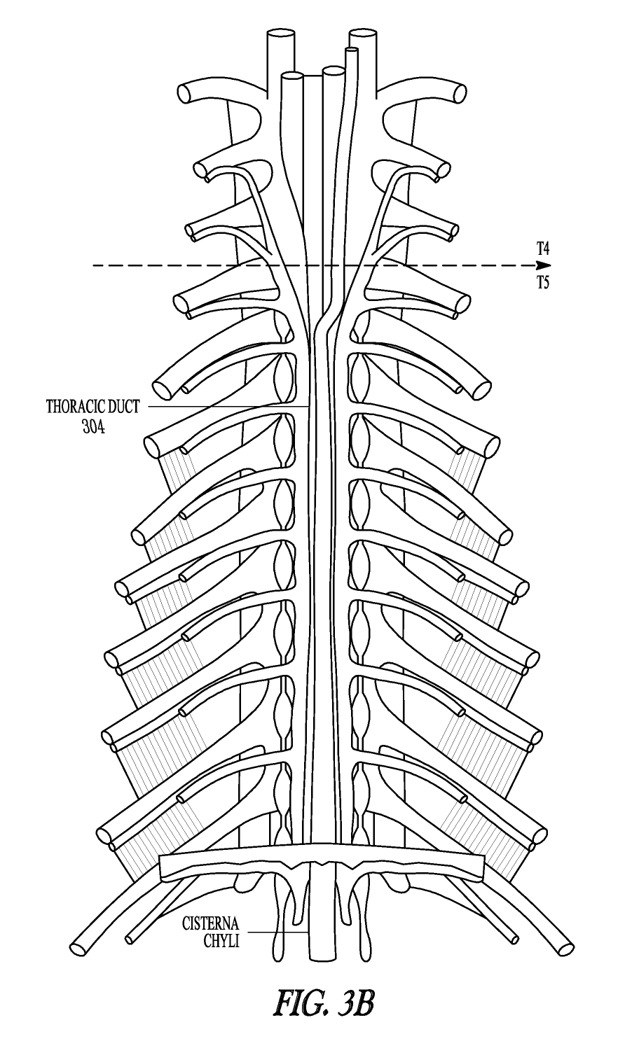

FIGS. 3A-3C illustrate anatomy proximate to the thoracic duct. With reference to FIGS. 3A-3B, the right vagus nerve 311 and the left vagus nerve 312 run closely past the thoracic duct near the T4/T5 region on the spine. The left vagus nerve is also typically near the thoracic duct in the cervical C5-C7 range. Various embodiments target the right vagus nerve 311 and/or the left vagus nerve 312 with translymphatic stimulation using electrode(s) within the lymphatic duct. FIG. 3C illustrates a cross-sectional view that includes, among other things, representations for the thoracic duct 304 and the vagus nerves 311/312, as well as the spine 313, heart 314 and rib of a human.

The spinal cord is nerve tissue that carries neural messages between the brain and parts of the body. Nerve roots branch off and exit the spine on both sides through spaces between the vertebra. The spinal column includes cervical, thoracic and lumbar areas. Vertebrae form the building blocks of the spinal column and protect the spinal cord. T1-T5 are the uppermost (cranial) portion of the thoracic area of the spinal column. Projections from T1-T5 innervate the heart. The spinal projections from T1-T5 are sympathetic. Increased efferent sympathetic activity increases heart rate and contractility. Afferent for the heart tissue also go throughout spinal segments T1-T5. Various embodiments target the T1-T5 region for cardiovascular disease applications. Other regions may be targeted for other applications (e.g. treatment for hypertension, diabetes, obesity, etc.).

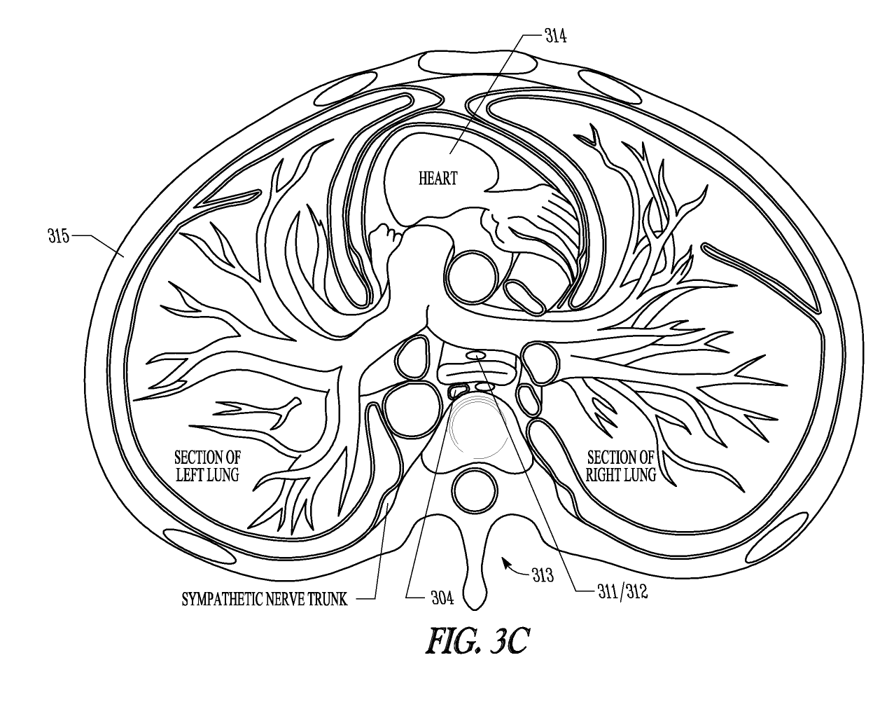

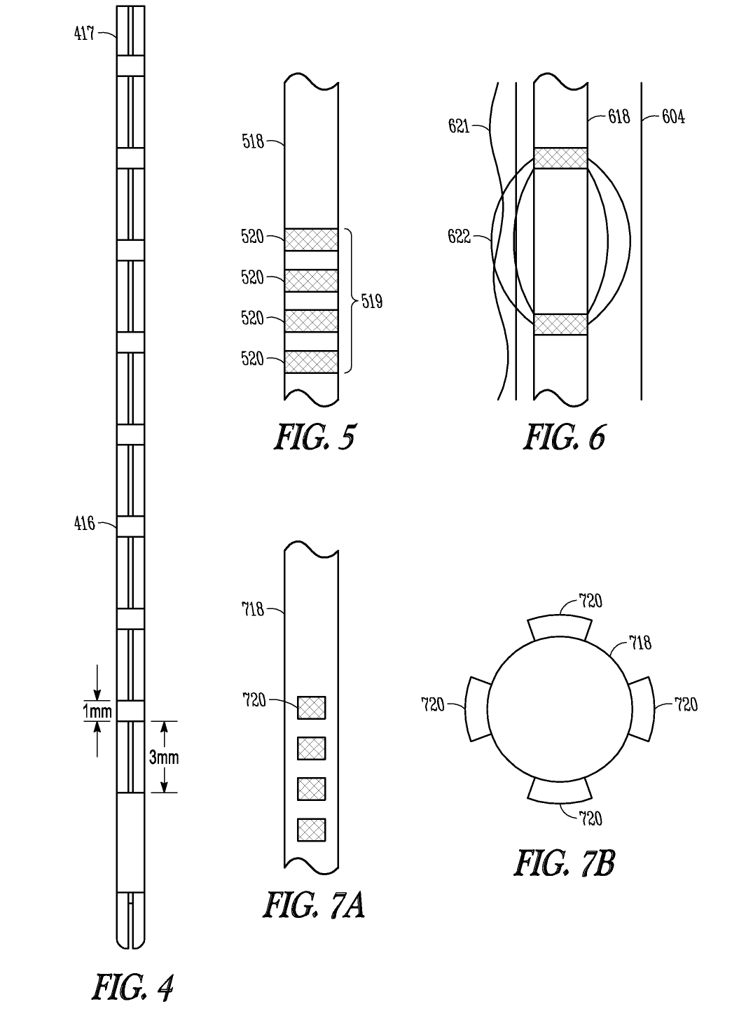

FIGS. 4-7B illustrate various lead embodiments. FIG. 4 illustrates an embodiment of a lead with a plurality of contacts. According to various lead embodiments, a focused therapy is delivered using a plurality of contacts and independent current sources for each contact to sculpt current to precisely reach desired nerve fibers. The independent current control at each contact eliminates the need to switch power on and off during programming to deliver smooth and rapid stimulation programming. Some embodiments provide two columns of tightly spaced contacts to sculpt current in three dimensions. Some lead embodiments use individually insulated multi-filar cables, where each contact is electrically connected to the pulse generator using multiple conductors. The tightly spaced contacts and the independent current controls can be used to overcome unique patient anatomies and impedance changes caused by scarring, and to maintain effective therapy over time.

A neural stimulation test routine can be implemented during the implantation procedure, or can be intermittently implemented during use to assess neural stimulation efficacy for electrode subsets of a plurality of electrodes to identify a desired electrode subset for use in delivering the neural stimulation therapy to elicit a desired response. Each electrode subset of the plurality of electrodes includes at least one electrode. The electrode subsets can include various combinations of electrodes selected from the plurality of electrodes, including all of the electrodes in the plurality of electrodes.

A number of electrode configurations can be used. The illustrations included herein are provided as examples, and are not intended to be an exhaustive listing of possible configurations.

FIG. 5 illustrates an embodiment of a tether or lead 518 with an electrode region 519 that includes annular stimulation electrodes 520, according to various embodiments. Any one or combination of the annular stimulation electrodes can be used to deliver the neural stimulation.

FIG. 6 illustrates transluminal neural stimulation using electrodes within the thoracic duct, according to various embodiments. The figure illustrates a lumen 1138 (e.g. thoracic duct 604), a nerve 621 external to the lumen, and a flexible lead 618 within the lumen. The neural stimulation generates an electrical field 622 between the electrodes that extends past the lumen wall to the nerve.

FIGS. 7A and 7B illustrate an embodiment of a lead 718 with stimulation electrodes 720, where the illustrated electrodes do not circumscribe the lead. Thus, a subset of the illustrated electrodes can be selected to provide directional stimulation. A neural stimulation test routine can cycle through the available electrodes for use in delivering the neural stimulation to determine which subset of electrodes are facing toward the neural target.

According to various embodiments, if an efficacy of a first electrode configuration is lower than a threshold, the system switches to a second electrode configuration to deliver neural stimulation. In some embodiments, if an efficacy of a first electrode configuration is lower than a threshold, the system switches to a second electrode configuration by removing an electrode to deliver neural stimulation. In some embodiments, if an efficacy of the first electrode configuration is lower than a threshold, the system switches to a second electrode configuration by adding an electrode to deliver neural stimulation. Other embodiments of electrode configurations that are adapted to stimulate a neural target are within the scope of this disclosure. In various embodiments, switching electrode configuration changes stimulation from bipolar to unipolar. In various embodiments, switching electrode configuration changes stimulation among a unipolar stimulation, a bipolar stimulation, or a multipolar stimulation. Various embodiments use current steering to change the direction of current flow. For example, in situations where current flows from both a first and second electrode to a third electrode, the stimulation parameters can be adjusted, such as by changing the applied potential between electrodes, to change the stimulation intensity and location between the first and third electrodes and between the second and third electrodes.

There is a high degree of individual anatomical variability with these structures. Thus, various embodiments provide an implantation technique with an optimization procedure. The optimization procedure may involve physically moving the electrodes or electronic repositioning the stimulation vectors in the general region until the desired effect is observed. The thoracic duct may be instrumented by introducing the electrodes into the subclavian vein and advancing to the thoracic duct ostium. The lead is advanced retrograde into the thoracic duct and positioned typically in the arch or ascending thoracic duct. The sympathetic stimulation electrodes may be positioned deeper in the thoracic duct in the C1-C8 and T1-T6 regions.

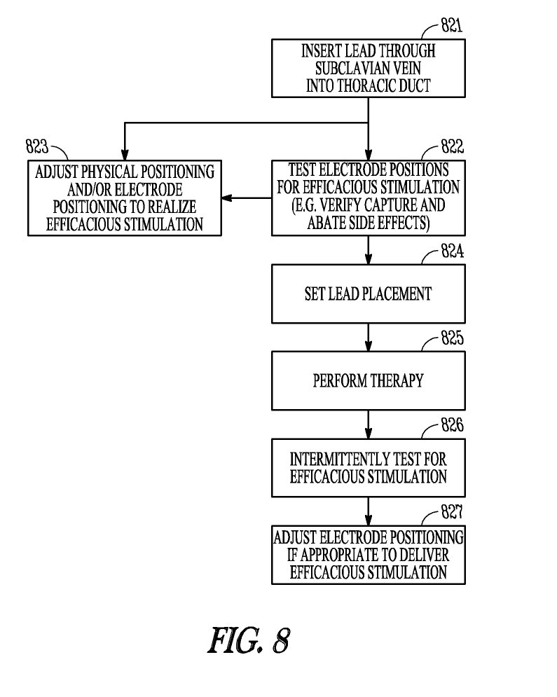

FIG. 8 illustrates a method embodiment to implant the spinal cord stimulation lead to establish and maintain efficacious stimulation therapy. At 821, a lead is inserted into the thoracic duct. At 822, the electrode positions are tested to determine if the electrode positions provide efficacious stimulation. For example, some embodiments monitor one or more physiological parameters to verify capture of the neural target (e.g. parasympathetic and sympathetic nerves). The present subject matter is capable of selectively stimulating or targeting only the parasympathetic nerves and/or selectively stimulating the sympathetic nerves. Some embodiments monitor one or more physiological parameter to abate potential unintended responses to the neural stimulation. If unable to verify capture or if undesired side effects are present during the implantation process, the process adjusts the physical positioning and/or the electronic positioning in an effort to realize efficacious stimulation, as represented at 823. The physical repositioning involves physically moving (e.g. pushing, pulling, rotating) the lead. The electronic repositioning involves selecting various combinations of electrodes to adjust the direction and position of the electric stimulation field. Electronic repositioning can be performed as part of an automatic process, where a device cycles through available electrode combinations (and stimulation intensity) until the desired efficacy is realized. Electronic repositioning can be controlled by a technician during the implant procedure. Some embodiments use a combination of technician control of potential configurations, and an automatic test routine.

When efficacious stimulation is detected, the physical lead placement is set at 824. A proximal end of the lead is connected to an implantable pulse generator. At 825, therapy is delivered using the implanted lead and the implanted pulse generator. At 826, the implanted pulse generator intermittently tests for efficacious stimulation to verify capture and/or abate side effects of the stimulation. If appropriate, the electronic positioning is adjusted to deliver efficacious stimulation, as illustrated at 827. This electronic repositioning can be performed automatically, controlled by a technician using a programmer, or a combination thereof.

FIGS. 9-18 illustrate various algorithms that can be programmed into the implantable device to control the translymphatic stimulation of the sympathetic and parasympathetic nerves.

FIG. 9 illustrates an embodiment where a programmable neural stimulator is programmed to chronically deliver neural stimulation pulses to chronically inhibit sympathetic activity in the sympathetic nerves branching from the spinal cord, and to intermittently deliver neural stimulation pulses to intermittently increase parasympathetic activity in the parasympathetic nerves branching from the spinal cord. The intermittent vagal stimulation enhances the effect of the sympathetic inhibition.

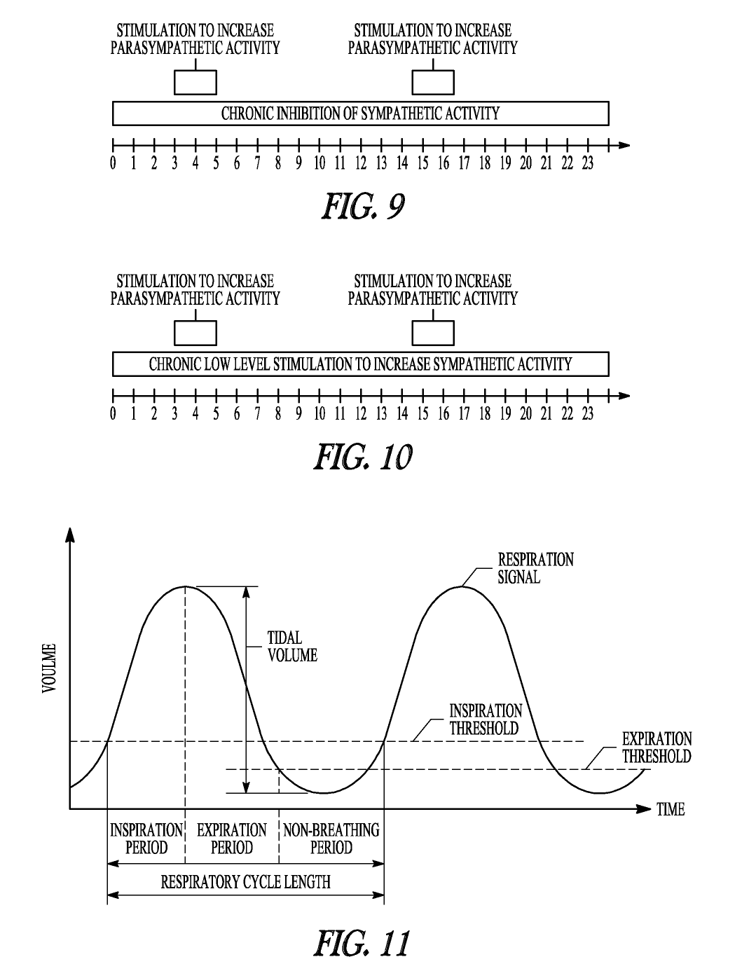

FIG. 10 illustrates an embodiment where a programmable neural stimulator is programmed to chronically deliver neural stimulation pulses to increase sympathetic activity in the sympathetic nerves branching from the spinal cord, and intermittently or chronically deliver neural stimulation pulses to increase parasympathetic activity in the parasympathetic nerves branching from the spinal cord. An embodiment chronically-delivers low-level sympathetic activation to enhance the impact of vagal stimulation through a vagal-sympathetic accentuated antagonism effect.

In an embodiment, the system includes a respiratory sensor, and the programmable neural stimulator is programmed to time delivery of neural stimulation pulses to decrease sympathetic activity during the inspiratory phase, and time delivery of neural stimulation pulses to increase parasympathetic activity during the expiratory phase. The respiration sensor can be used to guide the neural stimulation to block sympathetic activity during the inspiratory phase when sympathetic activity is intrinsically high, and to stimulate the vagus nerve during an expiratory phase to enhance the parasympathetic activity.

FIG. 11 is an illustration of a respiratory signal indicative of respiratory cycles and respiratory parameters including respiratory cycle length, inspiration period, expiration period, non-breathing period, and tidal volume. The inspiration period starts at the onset of the inspiration phase of a respiratory cycle, when the amplitude of the respiratory signal rises above an inspiration threshold, and ends at the onset of the expiration phase of the respiratory cycle, when the amplitude of the respiratory cycle peaks. The expiration period starts at the onset of the expiration phase and ends when the amplitude of the respiratory signal falls below an expiration threshold. The non-breathing period is the time interval between the end of the expiration phase and the beginning of the next inspiration phase. The tidal volume is the peak-to-peak amplitude of the respiratory signal.

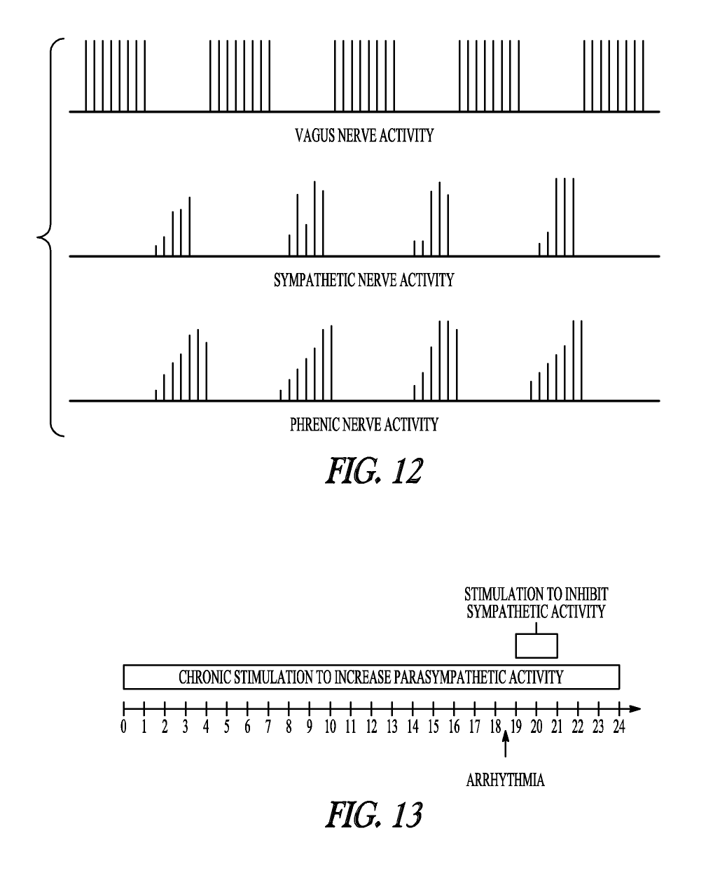

FIG. 12 illustrates the relationship between respiration, as illustrated by phrenic nerve activity, and both sympathetic nerve activity and vagus nerve activity. As illustrated, sympathetic nerve activity is most active during periods where the phrenic nerve activity is active, and parasympathetic nerve activity is most active during periods when the phrenic nerve activity is inactive.

According to some embodiments, timing is provided to deliver neural stimulation pulses to the first electrode region to decrease sympathetic activity during the inspiratory phase and to deliver neural stimulation pulses to the second electrode region to increase parasympathetic activity during the expiratory phase. For some embodiments, timing is provided to deliver neural stimulation pulses to the first electrode region to decrease sympathetic activity during the inspiratory phase, and deliver neural stimulation pulses to the second electrode region to increase parasympathetic activity during the inspiratory phase. For some embodiments, neural stimulation pulses is delivered to the first electrode region to chronically decrease sympathetic activity, and timing is provided to deliver neural stimulation pulses to the second electrode region to the respiratory cycle to intermittently increase parasympathetic activity. According to various embodiments, neural stimulation pulses is provided to the second electrode region to chronically increase parasympathetic activity, and timing is provided to deliver neural stimulation pulses to the first electrode region to the respiratory cycle to intermittently decrease sympathetic activity.

The respiratory signal is a physiologic signal indicative of respiratory activities. In various embodiments, the respiratory signal includes any physiology signal that is modulated by respiration. In one embodiment, the respiratory signal is a transthoracic impedance signal sensed by an implantable impedance sensor. In another embodiment, the respiratory signal is extracted from a blood pressure signal that is sensed by an implantable pressure sensor and includes a respiratory component. In another embodiment, the respiratory signal is sensed by an external sensor that senses a signal indicative of chest movement or lung volume. According to various embodiments, peaks of a respiratory signal are detected as respiratory fiducial points. A delay interval starts upon the detection of each of peaks. A burst of neural stimulation pulses is delivered to a nerve such as the vagus nerve when delay interval expires. In various other embodiments, onset points of the inspiration phases, ending points of the expiration phases, or other threshold-crossing points are detected as the respiratory fiducial points. A respiration-controlled neural stimulation circuit includes a stimulation output circuit and a controller that includes a respiratory signal input, a synchronization module, and a stimulation delivery controller. The respiratory signal input receives the respiratory signal indicative of respiratory cycles and respiratory parameters, and the synchronization module synchronizes the delivery of the neural stimulation pulses to the respiratory cycles. A respiratory fiducial point detector detects predetermined-type respiratory fiducial points in the respiratory signal, and a delay timer times a delay interval starting with each of the detected respiratory fiducial points. The stimulation delivery controller causes the stimulation output circuit to deliver a burst of the neural stimulation pulses when the delay interval expires.

FIG. 13 illustrates an embodiment where a programmable neural stimulator is programmed to implement an anti-arrhythmia therapy by delivering neural stimulation pulses to decrease sympathetic activity in the sympathetic nerves branching from the spinal cord if the arrhythmia detector detects the cardiac arrhythmia, and implement a chronic heart failure therapy by delivering neural stimulation pulses to chronically increase parasympathetic activity in the parasympathetic nerves branching from the spinal cord. This embodiment can be combined with various cardiac rhythm management devices (e.g. implantable cardioverter/defibrillator) that detect and treat arrhythmias.

In an embodiment, the programmable neural stimulator is programmed to deliver neural stimulation pulses to increase sympathetic activity in the sympathetic nerves branching from the spinal cord, deliver neural stimulation pulses to increase parasympathetic activity in the parasympathetic nerves branching from the spinal cord, and control timing of the neural stimulation pulses to intermittently increase both sympathetic and parasympathetic activity, and to follow increased sympathetic activity with increased parasympathetic activity. For example, sympathetic stimulation is delivered for a period of time. After the sympathetic stimulation ends, there is an intrinsic parasympathetic reflex response, which is augmented using vagal stimulation. This can be considered a type of conditioning therapy, also referred to as an intermittent stress therapy.

Some medical device embodiments stimulate a sympathetic neural target to provide the physical conditioning therapy, some medical device embodiments inhibit a parasympathetic neural target to provide physical conditioning therapy, and some medical device embodiments provide both sympathetic stimulation and parasympathetic inhibition for a physical conditioning therapy. Various embodiments provide a programmable pulse generator to deliver intermittent short periods of sympathetic stimulation and/or parasympathetic inhibition to mimic the effects of physical training. For example, the physical conditioning provided by the sympathetic stimulation and/or parasympathetic inhibition can occur on a daily basis for about 30 minutes/day. The therapy is of a relatively short duration. Embodiments provide therapy on the order of 2 hours or less. The physical conditioning therapy provided by the neural stimulation device can be programmed to correlate to a suitable exercise regimen for the patient. For example, the above-identified 30 minutes/day of neural stimulation can correspond to 30 minutes/day of walking. In another embodiment, by way of example, the present subject matter can provide physical conditioning therapy that corresponds to an every other day exercise regimen. In an embodiment, a patient or health-care provider controls the times when the therapy is initiated and terminated. Safety measures can be provided to prevent therapies of excessive duration. Closed-loop feedback of a physiological variable can be used to acutely titrate the therapy to achieve a desired response (e.g. to achieve and maintain a target heart rate zone during exercise or achieve a desired heart rate profile in which the heart rate increases and decreases) or abruptly terminate the therapy when the physiological response is adverse or otherwise indicates that the patient is not tolerating the therapy. The feedback can be used to adjust the intensity of the sympathetic stimulation/parasympathetic inhibition by appropriately adjusting the frequency and duration of the periods of sympathetic stimulation, and/or adjusting stimulation parameters.

Embodiments of the present subject matter provide heart failure therapy using physical conditioning. However, physical conditioning via sympathetic stimulation/parasympathetic inhibition is applied to any patient who may benefit from physical conditioning, but is unable to tolerate physical exercise. The present subject matter can be incorporated as a stand-alone neural stimulator, or integrated into an existing CRM device for comprehensive heart failure therapy, for example.

It is generally accepted that physical activity and fitness improve health and reduce mortality. Studies have indicated that aerobic training improves cardiac autonomic regulation, reduces heart rate and is associated with increased cardiac vagal outflow. A baseline measurement of higher parasympathetic activity is associated with improved aerobic fitness. Exercise training intermittently stresses the system and increases the sympathetic activity during the stress. However, when an exercise session ends and the stress is removed, the body rebounds in a manner that increases baseline parasympathetic activity and reduces baseline sympathetic activity.

Physical training stimulates the β₁-receptors of cardiac myocytes, which is a result of sympathetic stimulation. Short periods of exercise (e.g. less than 1-2 hours) result in an increase of β₁-receptor activity. On the other hand, periods of exercise longer than 2 hours can cause a reduction in β₁-receptor activity. Physical conditioning can be considered to be a repetitive, high-level exercise that occurs intermittently over time. The present subject matter mimics the effects of physical conditioning with sympathetic nerve stimulation and/or parasympathetic nerve inhibition.

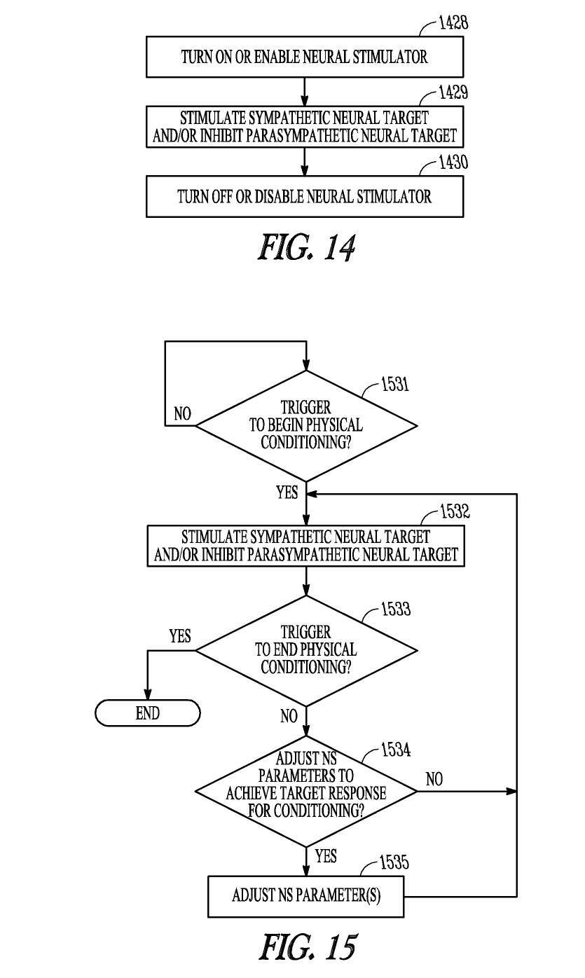

FIG. 14 illustrates a method for providing physical conditioning, according to various embodiments of the present subject matter. A neural stimulator (understood to include devices that apply electrical stimulation that stimulates nerve traffic and/or inhibits nerve traffic) is turned on or otherwise enabled at 1428. At 1429, the device stimulates a sympathetic neural target, inhibits a parasympathetic neural target, or both stimulates a sympathetic neural target and inhibits a parasympathetic neural target. At 1430, the device is turned off or otherwise disables the neural stimulator. In various external device embodiments, for example, the device includes a switch capable of being actuated by the patient or other person (e.g. physician) to turn the external device on and off. In various internal device embodiments, for example, the device is turned on and off through a wireless link. Examples of such wireless links include a magnetic field, and communications through induction, RF or ultrasound. Various embodiments provide user-initiated physical conditioning therapy, where a user “turns on” the therapy, which runs for a preprogrammed time. Various embodiments provide user-terminated physical conditioning therapy, where a programmed therapy is prematurely terminated by the user, regardless of whether the user initiated the physical conditioning therapy. Various embodiments provide user-titrated physical conditioning therapy, where the intensity and/or duration of the physical conditioning therapy can be increased or decreased by the user. The user can be a patient, a physician or other person. These user-initiated, user-terminated, and user-titrated embodiments can be internal or external devices. Various embodiments provide the ability for a user to perform all three functions (initiate, terminate and titrate), or any combination of two or more of these functions. An internal device embodiment uses an internal timer to turn the device on and off. A pre-programmed schedule can control the on-time and off-time of the therapy. Other events can be used to either enable or disable the programmed on-time and off-time. For example, the programmed therapy can be enabled if the heart rate is within a predetermined zone, if the systolic blood pressure is within a predetermined zone, and/or the respiration rate is within a predetermined zone. A programmed therapy can be disabled or terminated if the heart rate is over a predetermined threshold, the systolic blood pressure is over a predetermined threshold, and/or the respiration rate is over a predetermined threshold.

FIG. 15 illustrates a method for providing physical conditioning, according to various embodiments of the present subject matter. At 1531, it is determined whether a trigger has been received to begin physical conditioning. When the trigger is detected, a sympathetic neural target is stimulated and/or a parasympathetic neural target is inhibited at 1532. At 1533, it is determined whether a trigger to end the physical conditioning has been received. Various implantable device embodiments are triggered by an external signal controlled by a physician or patient. A device embodiment uses a timer to turn the device on and off. A pre-programmed schedule can control the on-time and off-time of the therapy. Other events can be used to either enable or disable the programmed on-time and off-time. Various sensor feedback can be used to enable and/or disable the therapy. For example, the programmed therapy can be enabled if the heart rate is within a predetermined zone, if the systolic blood pressure is within a predetermined zone, and/or the respiration rate is within a predetermined zone. A programmed therapy can be disabled or terminated if the heart rate is over a predetermined threshold, the systolic blood pressure is over a predetermined threshold, and/or the respiration rate is over a predetermined threshold. If the trigger to end the therapy has not been received, it is determined at 1534 whether to adjust the neural stimulation parameters to achieve a target response for the conditioning therapy. Adjustable neural stimulation parameters include, but are not limited to, a stimulation duration as well as an amplitude, frequency, pulse width, morphology, and burst frequency of the neural stimulation signal. These parameters can be appropriately increased or decreased to obtain a desired change in the intensity of the neural stimulation/inhibition. Examples of target responses include a target heart rate range or target blood pressure range or respiratory rate for a period of time. If it is determined at 1534 to adjust the parameters, the process proceeds to 1535 to adjust the parameter(s) and returns to 1532; and if it is determined that the parameters will not be adjusted, the process returns from 1534 to 1532. Various embodiments provide target range(s) as programmable parameters, and various embodiments automatically adjust the intensity of the neural stimulation/inhibition to maintain a sensed physiological parameter (e.g. heart rate) within the target range. Various embodiments provide means for manually adjusting the intensity based on a sensed physiological parameter.

A physical conditioning therapy can be applied as therapy for heart failure. Examples of other physical conditioning therapies include therapies for patients who are unable to exercise. For example, physical conditioning using sympathetic stimulation/parasympathetic inhibition for a bed-bound, post-surgical patient in a hospital may enable the patient to maintain strength and endurance until such time that the patient is able to exercise again. By way of another example, a morbidly obese patient may be unable to exercise, but may still benefit from the physical conditioning therapy. Furthermore, patients with injuries such as joint injuries that prevent them from performing their normal physical activities may benefit from the physical conditioning therapy.

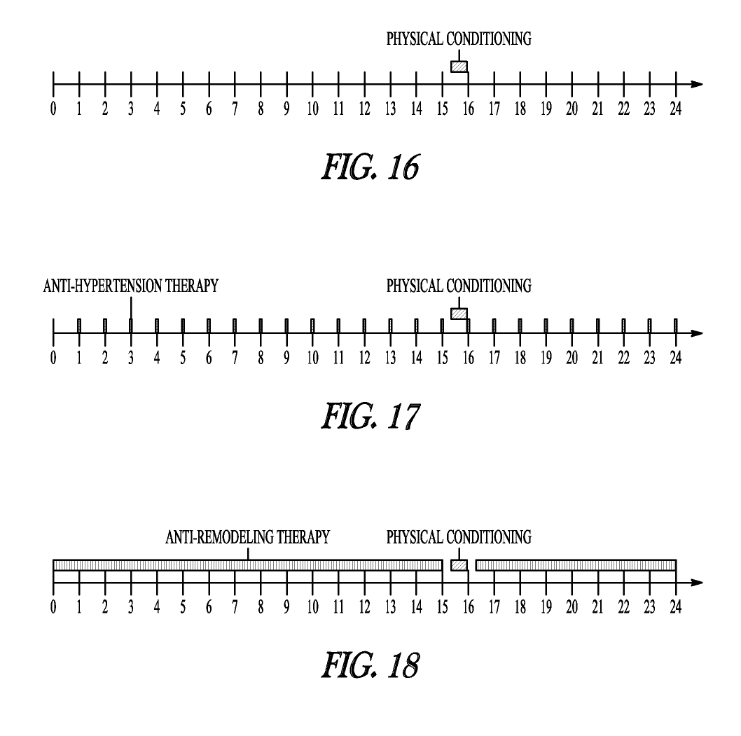

FIG. 16 illustrates a physical conditioning therapy using sympathetic stimulation and/or parasympathetic inhibition, according to various embodiments of the present subject matter, and FIGS. 17-18 illustrate examples of therapy protocols that combine or integrate sympathetic stimulation and/or parasympathetic inhibition associated with physical conditioning with therapies that use parasympathetic stimulation and/or sympathetic inhibition, according to various embodiments of the present subject matter. The time line is divided into 24 intervals, such as may be used to illustrate hours in a day. The illustrated therapies on the time line are intended as an example. Other therapy regimens can be implemented. In FIG. 16, it is illustrated that a physical conditioning therapy is applied for a short duration. This therapy is applied intermittently in some embodiments. Some embodiments apply the physical conditioning in a periodic manner (e.g. daily or every other day). For example, some embodiments apply the stimulation to mimic an exercise regimen (e.g. walk 5 times per week for 30 minutes and maintain a heart rate within a target range). Various embodiments provide the total therapy for the day (e.g. 30 minutes per day) in increments (e.g. 5 minutes of therapy provided 6 times per day) The physical conditioning involves sympathetic stimulation, parasympathetic inhibition, or both sympathetic stimulation and parasympathetic inhibition to intermittently stress the patient. In contrast, an anti-hypertension therapy, for example, applies parasympathetic stimulation, sympathetic inhibition, or both parasympathetic stimulation and sympathetic inhibition. The anti-hypertension therapy can be applied intermittently or periodically (e.g. 5 minutes every hour or 5 seconds every minute). As illustrated generally in FIG. 17, the application of the physical conditioning is timed to occur between anti-hypertension therapy. An anti-remodeling therapy also applies parasympathetic stimulation, sympathetic inhibition, or both parasympathetic stimulation and sympathetic inhibition. The anti-remodeling therapy can be provided on a more continuous basis. As illustrated generally in FIG. 18, the anti-remodeling therapy can be interrupted to provide a window of time in which to provide the physical conditioning therapy. Some embodiments are able to provide parasympathetic stimulation and inhibition at the same site selectable by varying, for example, the frequency of stimulation or polarity of stimulation. Some embodiments are able to provide sympathetic stimulation and inhibition at the same site selectable by varying, for example, the frequency of stimulation or polarity of stimulation. Some embodiments are able to simultaneously provide a local parasympathetic response at a first location and a local sympathetic response in another location.

Various embodiments of the present subject matter are used to deliver a heart failure therapy. The status of the heart failure can be determined in a number of ways. The heart failure status can be used by a clinician to assess the status of the heart failure and the effectiveness of the treatment (where a chronically-implanted device measures and stores parameters used to assess heart failure status), or can be used by the implanted device as feedback for a closed-loop therapy system. Examples of parameters that can be used to determine a HF status include heart rate variability (HRV), heart rate turbulence (HRT), heart sounds, electrogram features, activity, respiration, and pulmonary artery pressure. These parameters are briefly discussed below.

Respiration parameters, for example, can be derived from a minute ventilation signal and a fluid index can be derived from transthoracic impedance. For example, decreasing thoracic impedance reflects increased fluid buildup in lungs, and indicates a progression of heart failure. Respiration can significantly vary a minute ventilation. The transthoracic impedance can be totaled or averaged to provide a indication of fluid buildup.

Heart Rate Variability (HRV) is one technique that has been proposed to assess autonomic balance. HRV relates to the regulation of the sinoatrial node, the natural pacemaker of the heart by the sympathetic and parasympathetic branches of the autonomic nervous system. An HRV assessment is based on the assumption that the beat-to-beat fluctuations in the rhythm of the heart provide us with an indirect measure of heart health, as defined by the degree of balance in sympathetic and vagus nerve activity. The time interval between intrinsic ventricular heart contractions changes in response to the body's metabolic need for a change in heart rate and the amount of blood pumped through the circulatory system. For example, during a period of exercise or other activity, a person's intrinsic heart rate will generally increase over a time period of several or many heartbeats. However, even on a beat-to-beat basis, that is, from one heart beat to the next, and without exercise, the time interval between intrinsic heart contractions varies in a normal person. These beat-to-beat variations in intrinsic heart rate are the result of proper regulation by the autonomic nervous system of blood pressure and cardiac output; the absence of such variations indicates a possible deficiency in the regulation being provided by the autonomic nervous system. One method for analyzing HRV involves detecting intrinsic ventricular contractions, and recording the time intervals between these contractions, referred to as the R-R intervals, after filtering out any ectopic contractions (ventricular contractions that are not the result of a normal sinus rhythm). This signal of R-R intervals is typically transformed into the frequency-domain, so that its spectral frequency components can be analyzed and divided into low and high frequency bands. The HF band of the R-R interval signal is influenced only by the parasympathetic/vagal component of the autonomic nervous system. The LF band of the R-R interval signal is influenced by both the sympathetic and parasympathetic components of the autonomic nervous system. Consequently, the ratio LF/HF is regarded as a good indication of the autonomic balance between sympathetic and parasympathetic/vagal components of the autonomic nervous system. An increase in the LF/HF ratio indicates an increased predominance of the sympathetic component, and a decrease in the LF/HF ratio indicates an increased predominance of the parasympathetic component. For a particular heart rate, the LF/HF ratio is regarded as an indication of patient wellness, with a lower LF/HF ratio indicating a more positive state of cardiovascular health. A spectral analysis of the frequency components of the R-R interval signal can be performed using a FFT (or other parametric transformation, such as autoregression) technique from the time domain into the frequency domain. Such calculations require significant amounts of data storage and processing capabilities. Additionally, such transformation calculations increase power consumption, and shorten the time during which the implanted battery-powered device can be used before its replacement is required.

Heart rate turbulence (HRT) is the physiological response of the sinus node to a premature ventricular contraction (PVC), consisting of a short initial heart rate acceleration followed by a heart rate deceleration. HRT has been shown to be an index of autonomic function, closely correlated to HRV. HRT is believed to be an autonomic baroreflex. The PVC causes a brief disturbance of the arterial blood pressure (low amplitude of the premature beat, high amplitude of the ensuing normal beat). This fleeting change is registered immediately with an instantaneous response in the form of HRT if the autonomic system is healthy, but is either weakened or missing if the autonomic system is impaired.

By way of example and not limitation, it has been proposed to quantify HRT using Turbulence Onset (TO) and Turbulence Slope (TS). TO refers to the difference between the heart rate immediately before and after a PVC, and can be expressed as a percentage. For example, if two beats are evaluated before and after the PVC, TO can be expressed as:

$$T O \% = \frac{( {RR}_{+ 1} + {RR}_{+ 2} ) - ( {RR}_{- 2} + {RR}_{- 1} )}{( {RR}_{- 2} + {RR}_{- 1} )} * 100.$$

RR₋₂ and RR₋₁ are the first two normal intervals preceding the PVC and RR₊₁ and RR₊₂ are the first two normal intervals following the PVC. In various embodiments, TO is determined for each individual PVC, and then the average value of all individual measurements is determined. However, TO does not have to be averaged over many measurements, but can be based on one PVC event. Positive TO values indicate deceleration of the sinus rhythm, and negative values indicate acceleration of the sinus rhythm. The number of R-R intervals analyzed before and after the PVC can be adjusted according to a desired application. TS, for example, can be calculated as the steepest slope of linear regression for each sequence of five R-R intervals. In various embodiments, the TS calculations are based on the averaged tachogram and expressed in milliseconds per RR interval. However, TS can be determined without averaging. The number of R-R intervals in a sequence used to determine a linear regression in the TS calculation also can be adjusted according to a desired application.

Benefits of using HRT to monitor autonomic balance include the ability to measure autonomic balance at a single moment in time. Additionally, unlike the measurement of HRV, HRT assessment can be performed in patients with frequent atrial pacing. Further, HRT analysis provides for a simple, non-processor-intensive measurement of autonomic balance. Thus, data processing, data storage, and data flow are relatively small, resulting in a device with less cost and less power consumption. Also, HRT assessment is faster than HRV, requiring much less R-R data. HRT allows assessment over short recording periods similar in duration to typical neural stimulation burst durations, such as on the order of tens of seconds, for example.

Distinguishable heart sounds include the following four heart sounds. The first heart sound (S₁), is initiated at the onset of ventricular systole and consists of a series of vibrations of mixed, unrelated, low frequencies. It is the loudest and longest of the heart sounds, has a decrescendo quality, and is heard best over the apical region of the heart. The tricuspid valve sounds are heard best in the fifth intercostal space, just to the left of the sternum, and the mitral sounds are heard best in the fifth intercostal space at the cardiac apex. S₁ is chiefly caused by oscillation of blood in the ventricular chambers and vibration of the chamber walls. The vibrations are engendered by the abrupt rise of ventricular pressure with acceleration of blood back toward the atria, and the sudden tension and recoil of the A-V valves and adjacent structures with deceleration of the blood by the closed A-V valves. The vibrations of the ventricles and the contained blood are transmitted through surrounding tissue and reach the chest wall where they may be heard or recorded. The intensity of S₁ is primarily a function of the force of the ventricular contraction, but also of the interval between atrial and ventricular systoles. If the A-V valve leaflets are not closed prior to ventricular systole, greater velocity is imparted to the blood moving toward the atria by the time the A-V valves are snapped shut by the rising ventricular pressure, and stronger vibrations result from this abrupt deceleration of the blood by the closed A-V valves. The second heart sound (S₂), which occurs on closure of the semi-lunar valves, is composed of higher frequency vibrations, is of shorter duration and lower intensity, and has a more “snapping” quality than the first heart sound. The second sound is caused by abrupt closure of the semi-lunar valves, which initiates oscillations of the columns of blood and the tensed vessel walls by the stretch and recoil of the closed valve. Conditions that bring about a more rapid closure of the semi-lunar valve, such as increases in pulmonary artery or aorta pressure (e.g., pulmonary or systemic hypertension), will increase the intensity of the second heart sound. In the adult, the aortic valve sound is usually louder than the pulmonic, but in cases of pulmonary hypertension, the reverse is often true. The third heart sound (S₃), which is more frequently heard in children with thin chest walls or in patients with rapid filling wave due to left ventricular failure, consists of a few low intensity, low-frequency vibrations. It occurs in early diastole and is believed to be due to vibrations of the ventricular walls caused by abrupt acceleration and deceleration of blood entering the ventricles on opening of the atrial ventricular valves. A fourth or atrial sound (S₄), consisting of a few low-frequency oscillations, is occasionally heard in normal individuals. It is caused by oscillation of blood and cardiac chambers created by atrial contraction. Accentuated S₃ and S₄ sounds may be indicative of certain abnormal conditions and are of diagnostic significance.

Thus, a heart sound can be used in determining a heart failure status. For example, a more severe HF status tends to be reflected in a larger S₃ amplitude.

Example of ECG features that can be extracted to provide an indicator of HF status include a QRS complex duration due to left bundle branch block, ST segment deviation, and a Q wave due to myocardial infarction. Any one or combination of these features can be used to provide the indicator of HF status. Other features can be extracted from the ECG.

Activity sensors can be used to assess the activity of the patient. Sympathetic activity naturally increases in an active patient, and decreases in an inactive patient. Thus, activity sensors can provide a contextual measurement for use in determining the autonomic balance of the patient, and thus the HF status of the patient. Various embodiments, for example, provide a combination of sensors to trend heart rate and/or respiration rate to provide an indicator of activity.

Two methods for detecting respiration involve measuring a transthoracic impedance and minute ventilation. Respiration can be an indicator of activity, and can provide an explanation of increased sympathetic tone that does not directly relate to a HF status. For example, it may not be appropriate to change a HF therapy due to a detected increase in sympathetic activity attributable to exercise.

Respiration measurements (e.g. transthoracic impedance) can also be used to measure Respiratory Sinus Arrhythmia (RSA). RSA is the natural cycle of arrhythmia that occurs through the influence of breathing on the flow of sympathetic and vagus impulses to the sinoatrial node. The rhythm of the heart is primarily under the control of the vagus nerve, which inhibits heart rate and the force of contraction. The vagus nerve activity is impeded and heart rate begins to increase when a breath is inhaled. When exhaled, vagus nerve activity increases and the heart rate begins to decrease. The degree of fluctuation in heart rate is also controlled significantly by regular impulses from the baroreceptors (pressure sensors) in the aorta and carotid arteries. Thus, a measurement of autonomic balance can be provided by correlating heart rate to the respiration cycle.

As identified above, high blood pressure can contribute to heart failure. Chronically high blood pressure, or a chronic blood pressure that trends higher, provides an indication of an increased likelihood of heart failure. Various embodiments use pulmonary artery pressure to approximate filling pressure. Filling pressure is a marker of preload, and preload is an indicator of heart failure status.

Various neural stimulation therapies can be integrated with various myocardial stimulation therapies. The integration of therapies may have a synergistic effect. Therapies can be synchronized with each other, and sensed data can be shared. A myocardial stimulation therapy provides a cardiac therapy using electrical stimulation of the myocardium. Some examples of myocardial stimulation therapies are provided below.

A pacemaker is a device which paces the heart with timed pacing pulses, most commonly for the treatment of bradycardia where the ventricular rate is too slow. If functioning properly, the pacemaker makes up for the heart's inability to pace itself at an appropriate rhythm in order to meet metabolic demand by enforcing a minimum heart rate. Implantable devices have also been developed that affect the manner and degree to which the heart chambers contract during a cardiac cycle in order to promote the efficient pumping of blood. The heart pumps more effectively when the chambers contract in a coordinated manner, a result normally provided by the specialized conduction pathways in both the atria and the ventricles that enable the rapid conduction of excitation (i.e., depolarization) throughout the myocardium. These pathways conduct excitatory impulses from the sino-atrial node to the atrial myocardium, to the atrio-ventricular node, and thence to the ventricular myocardium to result in a coordinated contraction of both atria and both ventricles. This both synchronizes the contractions of the muscle fibers of each chamber and synchronizes the contraction of each atrium or ventricle with the contralateral atrium or ventricle. Without the synchronization afforded by the normally functioning specialized conduction pathways, the heart's pumping efficiency is greatly diminished. Pathology of these conduction pathways and other inter-ventricular or intra-ventricular conduction deficits can be a causative factor in heart failure, which refers to a clinical syndrome in which an abnormality of cardiac function causes cardiac output to fall below a level adequate to meet the metabolic demand of peripheral tissues. In order to treat these problems, implantable cardiac devices have been developed that provide appropriately timed electrical stimulation to one or more heart chambers in an attempt to improve the coordination of atrial and/or ventricular contractions, termed cardiac resynchronization therapy (CRT). Ventricular resynchronization is useful in treating heart failure because, although not directly inotropic, resynchronization can result in a more coordinated contraction of the ventricles with improved pumping efficiency and increased cardiac output. Currently, a common form of CRT applies stimulation pulses to both ventricles, either simultaneously or separated by a specified biventricular offset interval, and after a specified atrio-ventricular delay interval with respect to the detection of an intrinsic atrial contraction or delivery of an atrial pace.

CRT can be beneficial in reducing the deleterious ventricular remodeling which can occur in post-MI and heart failure patients. Presumably, this occurs as a result of changes in the distribution of wall stress experienced by the ventricles during the cardiac pumping cycle when CRT is applied. The degree to which a heart muscle fiber is stretched before it contracts is termed the preload, and the maximum tension and velocity of shortening of a muscle fiber increases with increasing preload. When a myocardial region contracts late relative to other regions, the contraction of those opposing regions stretches the later contracting region and increases the preload. The degree of tension or stress on a heart muscle fiber as it contracts is termed the afterload. Because pressure within the ventricles rises rapidly from a diastolic to a systolic value as blood is pumped out into the aorta and pulmonary arteries, the part of the ventricle that first contracts due to an excitatory stimulation pulse does so against a lower afterload than does a part of the ventricle contracting later. Thus a myocardial region which contracts later than other regions is subjected to both an increased preload and afterload. This situation is created frequently by the ventricular conduction delays associated with heart failure and ventricular dysfunction due to an MI. The increased wall stress to the late-activating myocardial regions is most probably the trigger for ventricular remodeling. By pacing one or more sites in a ventricle near the infarcted region in a manner which may cause a more coordinated contraction, CRT provides pre-excitation of myocardial regions which would otherwise be activated later during systole and experience increased wall stress. The pre-excitation of the remodeled region relative to other regions unloads the region from mechanical stress and allows reversal or prevention of remodeling to occur.

Cardioversion, an electrical shock delivered to the heart synchronously with the QRS complex, and defibrillation, an electrical shock delivered without synchronization to the QRS complex, can be used to terminate most tachyarrhythmias. The electric shock terminates the tachyarrhythmia by simultaneously depolarizing the myocardium and rendering it refractory. A class of CRM devices known as an implantable cardioverter defibrillator (ICD) provides this kind of therapy by delivering a shock pulse to the heart when the device detects tachyarrhythmias. Another type of electrical therapy for tachycardia is anti-tachycardia pacing (ATP). In ventricular ATP, the ventricles are competitively paced with one or more pacing pulses in an effort to interrupt the reentrant circuit causing the tachycardia. Modern ICDs typically have ATP capability, and deliver ATP therapy or a shock pulse when a tachyarrhythmia is detected.

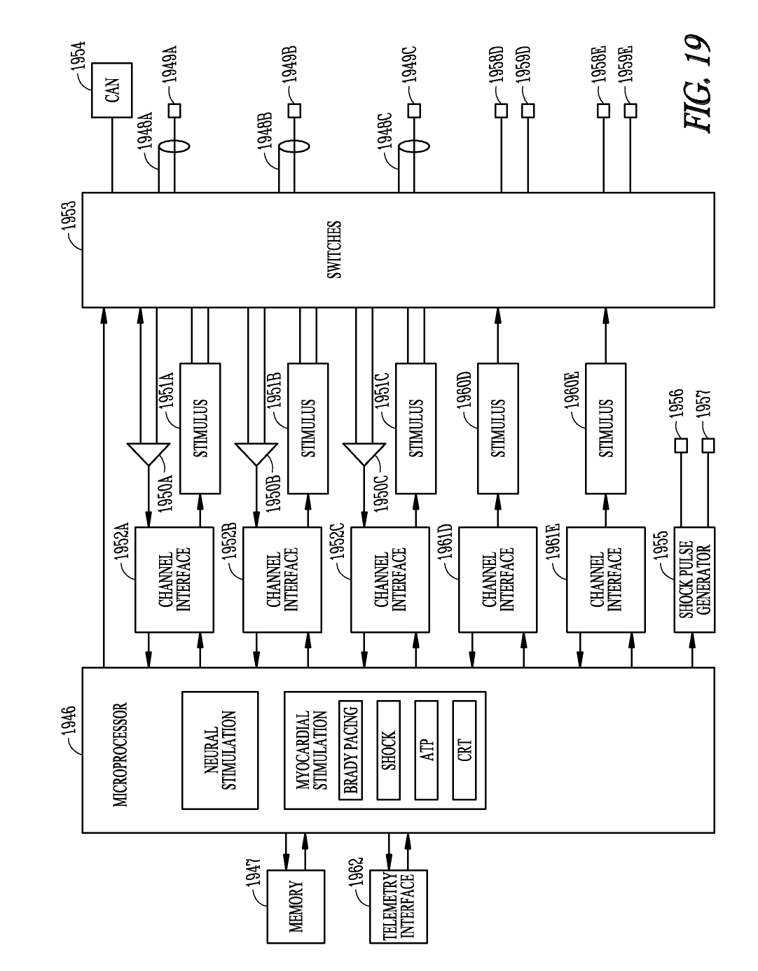

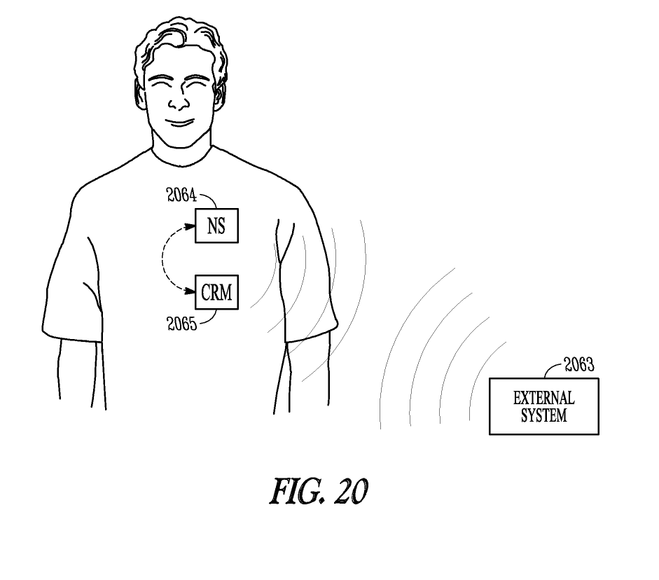

FIG. 19 shows a system diagram of an embodiment of a microprocessor-based implantable device, according to various embodiments. The controller of the device is a microprocessor 1946 which communicates with a memory 1947 via a bidirectional data bus. The controller could be implemented by other types of logic circuitry (e.g., discrete components or programmable logic arrays) using a state machine type of design, but a microprocessor-based system is preferable. As used herein, the term “circuitry” should be taken to refer to either discrete logic circuitry or to the programming of a microprocessor. Shown in the figure are three examples of sensing and pacing channels designated “A” through “C” comprising bipolar leads with ring electrodes 1948A-C and tip electrodes 1949A-C, sensing amplifiers 1950A-C, pulse generators 1951A-C, and channel interfaces 1952A-C. Each channel thus includes a pacing channel made up of the pulse generator connected to the electrode and a sensing channel made up of the sense amplifier connected to the electrode. The channel interfaces 1952A-C communicate bidirectionally with the microprocessor 1946, and each interface may include analog-to-digital converters for digitizing sensing signal inputs from the sensing amplifiers and registers that can be written to by the microprocessor in order to output pacing pulses, change the pacing pulse amplitude, and adjust the gain and threshold values for the sensing amplifiers. The sensing circuitry of the pacemaker detects a chamber sense, either an atrial sense or ventricular sense, when an electrogram signal (i.e., a voltage sensed by an electrode representing cardiac electrical activity) generated by a particular channel exceeds a specified detection threshold. Pacing algorithms used in particular pacing modes employ such senses to trigger or inhibit pacing. The intrinsic atrial and/or ventricular rates can be measured by measuring the time intervals between atrial and ventricular senses, respectively, and used to detect atrial and ventricular tachyarrhythmias.

The electrodes of each bipolar lead are connected via conductors within the lead to a switching network 1953 controlled by the microprocessor. The switching network is used to switch the electrodes to the input of a sense amplifier in order to detect intrinsic cardiac activity and to the output of a pulse generator in order to deliver a pacing pulse. The switching network also enables the device to sense or pace either in a bipolar mode using both the ring and tip electrodes of a lead or in a unipolar mode using only one of the electrodes of the lead with the device housing (can) 1954 or an electrode on another lead serving as a ground electrode. A shock pulse generator 1955 is also interfaced to the controller for delivering a defibrillation shock via a pair of shock electrodes 1956 and 1957 to the atria or ventricles upon detection of a shockable tachyarrhythmia.

Neural stimulation channels, identified as channels D and E, are incorporated into the device for delivering parasympathetic stimulation and/or sympathetic inhibition, where one channel includes a bipolar lead with a first electrode 1958D and a second electrode 1959D, a pulse generator 1960D, and a channel interface 1961D, and the other channel includes a bipolar lead with a first electrode 1958E and a second electrode 1959E, a pulse generator 1960E, and a channel interface 1961E. Other embodiments may use unipolar leads in which case the neural stimulation pulses are referenced to the can or another electrode. The pulse generator for each channel outputs a train of neural stimulation pulses which may be varied by the controller as to amplitude, frequency, duty-cycle, and the like. In this embodiment, each of the neural stimulation channels uses a lead which can be intravascularly disposed near an appropriate neural target. Other types of leads and/or electrodes may also be employed. A nerve cuff electrode may be used in place of an intravascularly disposed electrode to provide neural stimulation. In some embodiments, the leads of the neural stimulation electrodes are replaced by wireless links.

The figure illustrates a telemetry interface 1962 connected to the microprocessor, which can be used to communicate with an external device. The illustrated microprocessor 1946 is capable of performing neural stimulation therapy routines and myocardial stimulation routines. Examples of NS therapy routines include a heart failure therapy, an anti-hypertension therapy (AHT), anti-remodeling therapy (ART), and anti-arrhythmia therapy. Examples of myocardial therapy routines include bradycardia pacing therapies, anti-tachycardia shock therapies such as cardioversion or defibrillation therapies, anti-tachycardia pacing therapies (ATP), and cardiac resynchronization therapies (CRT).

The neural stimulation and cardiac rhythm management functions may be integrated in the same device, as generally illustrated in FIG. 19 or may be separated into functions performed by separate devices. FIG. 20 illustrates a system including an external device 2063, an implantable neural stimulator (NS) device 2064 and an implantable cardiac rhythm management (CRM) device 2065, according to various embodiments of the present subject matter. Various aspects involve a method for communicating between an NS device and a CRM device or other cardiac stimulator. In various embodiments, this communication allows one of the devices 2064 or 2065 to deliver more appropriate therapy (i.e. more appropriate NS therapy or CRM therapy) based on data received from the other device. Some embodiments provide on-demand communications. In various embodiments, this communication allows each of the devices to deliver more appropriate therapy (i.e. more appropriate NS therapy and CRM therapy) based on data received from the other device. The illustrated NS device and the CRM device are capable of wirelessly communicating with each other, and the external system is capable of wirelessly communicating with at least one of the NS and the CRM devices. For example, various embodiments use telemetry coils to wirelessly communicate data and instructions to each other. In other embodiments, communication of data and/or energy is by ultrasonic means. Rather than providing wireless communication between the NS and CRM devices, various embodiments provide a communication cable or wire, such as an intravenously-fed lead, for use to communicate between the NS device and the CRM device. In some embodiments, the external system functions as a communication bridge between the NS and CRM devices.

One of ordinary skill in the art will understand that, the modules and other circuitry shown and described herein can be implemented using software, hardware, and combinations of software and hardware. As such, the terms module and circuitry, for example, are intended to encompass software implementations, hardware implementations, and software and hardware implementations.

The methods illustrated in this disclosure are not intended to be exclusive of other methods within the scope of the present subject matter. Those of ordinary skill in the art will understand, upon reading and comprehending this disclosure, other methods within the scope of the present subject matter. The above-identified embodiments, and portions of the illustrated embodiments, are not necessarily mutually exclusive. These embodiments, or portions thereof, can be combined. In various embodiments, the methods are implemented using a computer data signal embodied in a carrier wave or propagated signal, that represents a sequence of instructions which, when executed by one or more processors cause the processor(s) to perform the respective method. In various embodiments, the methods are implemented as a set of instructions contained on a computer-accessible medium capable of directing a processor to perform the respective method. In various embodiments, the medium is a magnetic medium, an electronic medium, or an optical medium.

The above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A system for modulating autonomic neural activity in a body having a spinal cord, a subclavian vein and thoracic lymphatic vessels that include a thoracic duct and a right lymphatic duct, the system comprising:
at least one stimulation lead including at least a first electrode region and a second electrode region, the at least one stimulation lead being adapted to be fed through the subclavian vein into a desired thoracic lymphatic vessel to operationally position the first electrode region in the thoracic lymphatic vessel to stimulate sympathetic nerves branching from a first region of the spinal cord and to operationally position the second electrode region in the desired thoracic lymphatic vessel to stimulate sympathetic nerves branching from a second region of the spinal cord or stimulate parasympathetic nerves anatomically adjacent to the desired thoracic lymphatic vessel; and
a programmable neural stimulator programmed to deliver neural stimulation pulses to the first electrode region to modulate sympathetic activity in the sympathetic nerves branching from the first region of the spinal cord and to deliver neural stimulation pulses to the second electrode region to modulate sympathetic activity in the sympathetic nerves branching from the second region of the spinal cord or to modulate parasympathetic activity in the parasympathetic nerves anatomically adjacent to the desired thoracic lymphatic vessel.

2. The system of claim 1, wherein the at least one stimulation lead includes a third electrode region, and the at least one stimulation lead is adapted to be fed into the desired thoracic lymphatic vessel to operationally position the second electrode region in the thoracic lymphatic vessel to stimulate the sympathetic nerves branching from the second region of the spinal cord, and to operationally position the third electrode region in the thoracic lymphatic vessel to stimulate parasympathetic nerves anatomically adjacent to the thoracic lymphatic vessel.

3. The system of claim 1, wherein the sympathetic nerves includes sympathetic nerves branching from the C5-T5 region of the spinal cord.

4. The system of claim 1, wherein the parasympathetic nerves includes a vagus nerve positioned anatomically adjacent to the desired thoracic lymphatic vessel.

5. The system of claim 1, wherein the programmable neural stimulator is programmed to deliver neural stimulation pulses to the first electrode region to decrease sympathetic activity in the sympathetic nerves branching from the first region of the spinal cord and to deliver neural stimulation pulses to the first electrode region to increase sympathetic activity in the sympathetic nerves branching from the first region of the spinal cord.

6. The system of claim 1, wherein the programmable neural stimulator is programmed to deliver neural stimulation pulses to the second electrode region to decrease parasympathetic activity in the parasympathetic nerves and to deliver neural stimulation pulses to the second electrode region to increase parasympathetic activity in the parasympathetic nerves.

7. The system of claim 1, wherein the at least one stimulation lead is a single lead that includes the first and second electrode regions.

8. The system of claim 7, wherein the single lead is a telescoping lead adapted to adjust the distance between the first electrode region and the second electrode region in the lead.

9. The system of claim 1, wherein the programmable neural stimulator is programmed to:
chronically deliver neural stimulation pulses to the first electrode region to chronically inhibit sympathetic activity in the sympathetic nerves branching from the first region of the spinal cord; and
intermittently deliver neural stimulation pulses to the second electrode region to intermittently increase parasympathetic activity in the parasympathetic nerves.

10. The system of claim 1, wherein the programmable neural stimulator is programmed to:
chronically deliver neural stimulation pulses to the first electrode region to increase sympathetic activity in the sympathetic nerves branching from the first region of the spinal cord; and
intermittently or chronically deliver neural stimulation pulses to the second electrode region to increase parasympathetic activity in the parasympathetic nerves.

11. The system of claim 1, further comprising a respiratory sensor operationally connected to the neural stimulator and adapted for use to detect an inspiratory and expiratory phase of a respiration cycle, wherein the programmable neural stimulator is programmed to:
time delivery of neural stimulation pulses to the first electrode region to decrease sympathetic activity during the inspiratory phase; and
time delivery of neural stimulation pulses to the second electrode region to increase parasympathetic activity during the expiratory phase.

12. The system of claim 1, further comprising a respiratory sensor operationally connected to the neural stimulator and adapted for use to detect an inspiratory and expiratory phase of a respiration cycle, wherein the programmable neural stimulator is programmed to:
time delivery of neural stimulation pulses to the first electrode region to decrease sympathetic activity during the inspiratory phase; and
time delivery of neural stimulation pulses to the second electrode region to increase parasympathetic activity during the inspiratory phase.

13. The system of claim 1, further comprising a respiratory sensor operationally connected to the neural stimulator and adapted for use to detect an inspiratory and expiratory phase of a respiration cycle, wherein the programmable neural stimulator is programmed to:
deliver neural stimulation pulses to the first electrode region to chronically decrease sympathetic activity; and
time delivery of neural stimulation pulses to the second electrode region to the respiratory cycle to intermittently increase parasympathetic activity.

14. The system of claim 1, further comprising a respiratory sensor operationally connected to the neural stimulator and adapted for use to detect an inspiratory and expiratory phase of a respiration cycle, wherein the programmable neural stimulator is programmed to:
deliver neural stimulation pulses to the second electrode region to chronically increase parasympathetic activity; and
time delivery of neural stimulation pulses to the first electrode region to the respiratory cycle to intermittently decrease sympathetic activity.

15. The system of claim 1, further comprising an arrhythmia detector adapted for use in detecting a cardiac arrhythmia, wherein the programmable neural stimulator is programmed to:
implement an anti-arrhythmia therapy by delivering neural stimulation pulses to the first electrode region to decrease sympathetic activity in the sympathetic nerves if the arrhythmia detector detects the cardiac arrhythmia; and
implement a chronic heart failure therapy by delivering neural stimulation pulses to the second electrode region to chronically increase parasympathetic activity in the parasympathetic nerves.

16. The system of claim 1, wherein the programmable neural stimulator is programmed to:
deliver neural stimulation pulses to the first electrode region to increase sympathetic activity in the sympathetic nerves;
deliver neural stimulation pulses to the second electrode region to increase parasympathetic activity in the parasympathetic nerves; and
control timing of the neural stimulation pulses to intermittently increase both sympathetic and parasympathetic activity, and to follow increased sympathetic activity with increased parasympathetic activity.

17. The system of claim 1, wherein:
the first electrode region has a plurality of electrodes and the second electrode region has a plurality of electrodes; and
the programmable neural stimulator is programmed to implement a neural stimulation test routine to assess neural stimulation efficacy for electrode subsets in the first and second electrode regions to identify a desired electrode subset to elicit desired responses.

18. The system of claim 17, wherein the programmable neural stimulator is programmed to:
implement the neural stimulation test routine to assess neural stimulation efficacy for at least two stimulation vectors available for the desired electrode subset; or
assess neural stimulation efficacy for at least two neural stimulation intensity levels for the desired electrode subset.

19. A method for modulating autonomic neural activity in a body having a spinal cord, a subclavian vein and thoracic lymphatic vessels that include a thoracic duct and a right lymphatic duct, the method comprising:
implementing at least one programmed therapy using an implanted medical device to modulate autonomic neural activity, wherein implementing at least one programmed therapy includes:
increasing or decreasing sympathetic activity in sympathetic nerves branching from a first region of the spinal cord using a first electrode in a desired thoracic lymphatic vessel; and
increasing or decreasing parasympathetic activity in parasympathetic nerves adjacent to the desired thoracic lymphatic vessel or sympathetic activity in sympathetic nerves branching from a second region of the spinal cord using a second electrode in the desired thoracic lymphatic vessel.

20. The method of claim 19, wherein implementing at least one programmed therapy includes:
increasing or decreasing sympathetic activity in sympathetic nerves branching from a C5-T5 region of the spinal cord using the first electrode; and
increasing or decreasing parasympathetic activity in a vagus nerve using the second electrode in the thoracic duct.

21. The method of claim 19, wherein implementing at least one programmed therapy includes:
chronically delivering neural stimulation pulses to the first electrode to chronically inhibit sympathetic activity in the sympathetic nerves; and
intermittently delivering neural stimulation pulses to the second electrode to intermittently increase parasympathetic activity in the parasympathetic nerves.

22. The method of claim 19, wherein implementing at least one programmed therapy includes:
chronically delivering neural stimulation pulses to the first electrode to increase sympathetic activity in the sympathetic nerves; and
intermittently delivering neural stimulation pulses to the second electrode to intermittently increase parasympathetic activity in the parasympathetic nerves.

23. The method of claim 19, wherein implementing at least one programmed therapy includes:
chronically delivering neural stimulation pulses to the first electrode to increase sympathetic activity in the sympathetic nerves branching from the first region of the spinal cord; and
intermittently or chronically delivering neural stimulation pulses to the second electrode to increase parasympathetic activity in the parasympathetic nerves.

24. The method of claim 19, wherein implementing at least one programmed therapy includes:
detecting an inspiratory and expiratory phase of a respiration cycle;
timing delivery of neural stimulation pulses to the first electrode to decrease sympathetic activity during the inspiratory phase in the sympathetic nerves branching from the first region of the spinal cord; and
timing delivery of neural stimulation pulses to the second electrode to increase parasympathetic activity during the expiratory phase in the parasympathetic nerves.

25. The method of claim 19, wherein implementing at least one programmed therapy includes:
detecting a cardiac arrhythmia;
implementing an anti-arrhythmia therapy, wherein implementing the anti-arrhythmia therapy includes delivering neural stimulation pulses to the first electrode to decrease sympathetic activity in the sympathetic nerves if the arrhythmia detector detects the cardiac arrhythmia; and
implementing a chronic heart failure therapy, wherein implementing the chronic heart failure therapy includes delivering neural stimulation pulses to the second electrode to chronically increase parasympathetic activity in the parasympathetic nerves.

26. The method of claim 19, wherein implementing at least one programmed therapy includes:
delivering neural stimulation pulses to the first electrode to increase sympathetic activity in the sympathetic nerves;
delivering neural stimulation pulses to the second electrode to increase parasympathetic activity in the parasympathetic nerves; and
timing the neural stimulation pulses to intermittently increase both sympathetic and parasympathetic activity, and to follow increased sympathetic activity with increased parasympathetic activity.

27. A method for implanting a system for modulating both parasympathetic and sympathetic activity in a body having a spinal cord, a subclavian vein and thoracic lymphatic vessels that include a thoracic duct and a right lymphatic duct, the method comprising:
feeding at least one stimulation lead through the subclavian vein into the thoracic lymphatic vessels to operationally position a first electrode region in a desired thoracic lymphatic vessel to stimulate sympathetic nerves branching from a first region of the spinal cord and to operationally position a second electrode region in the thoracic duct to stimulate parasympathetic nerves adjacent to the desired thoracic lymphatic vessel;
implanting a programmable neural stimulator;
operationally attaching the programmable neural stimulator to the at least one stimulation lead to stimulate the sympathetic nerves and parasympathetic nerves; and
implementing a test routine to verify capture of the sympathetic nerves and parasympathetic nerves.

28. The method of claim 27, further comprising programming the programmable neural stimulator to generate stimulation pulses to increase or decrease sympathetic activity in the sympathetic nerves and to generate stimulation pulses to increase or decrease parasympathetic activity in the parasympathetic nerves.
